(19) United States
(12) Patent Application Publication
(10) Pub. No.: US 2009/0012301 A1
(43) Pub. Date: Jan. 8, 2009

(54) FEXOFENADINE CRYSTAL FORM AND PROCESSES FOR ITS PREPARATION THEREOF

(75) Inventors: Shlomit Wizel, Petah Tiqva (IL); Ilan Kor-Sade, Shoham (IL)

Correspondence Address: KENYON & KENYON LLP, ONE BROADWAY, NEW YORK, NY, 10004, US

(73) Assignee: Teva Pharmaceuticals USA, Inc.

(21) Appl. No.: 12/209,768

(22) Filed: Sep. 12, 2008

Related U.S. Application Data
(62) Division of application No. 11/238,508, filed on Sep. 28, 2005.
(60) Provisional application No. 60/613,688, filed on Sep. 28, 2004.

(51) Int. Cl. C07D 211/34

(52) U.S. Cl. 546/239

(57) ABSTRACT

Provided is a crystalline form of fexofenadine free base and processes for its preparation.

### CROSS REFERENCE TO RELATED APPLICATIONS

This application is a divisional application of U.S. patent application Ser. No. 11/238,508 filed Sep. 28, 2005, which claims the benefit of U.S. Provisional Application No. 60/613,688, filed Sep. 28, 2004, the contents of all of which are incorporated herein by reference.

### BACKGROUND OF THE INVENTION

4-[4-[4-(hydroxydiphenylmethyl)-1-piperidinyl]-1-hydroxybutyl]-α,α-dimethylbenzeneacetic acid of formula (I) (fexofenadine) is an H₁ receptor antagonist and a useful antihistaminic drug. It has low permeability into central nervous system tissues and weak antimuscarinic activity, causing it to have few systemic side effects.

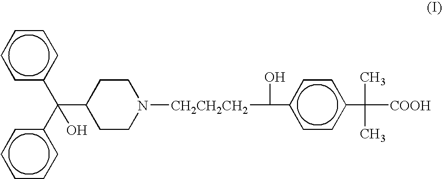

The antihistaminic activity of fexofenadine was first disclosed in U.S. Pat. No. 4,254,129, incorporated herein by reference. According to the '129 patent, fexofenadine can be prepared starting from ethyl α,α-dimethylphenyl acetate and 4-chlorobutyroyl chloride, which are reacted under Freidel-Crafts conditions. Chloride is displaced from the Freidel-Crafts product with α,α-diphenyl-4-piperidinemethanol to give 4-[4-[4-(hydroxydiphenylmethyl)-1-piperidinyl]-1-oxobutyl]-α,α-dimethylbenzeneacetate, which is isolated as its hydrochloride salt. The ketone is then reduced with PtO/H₂ and the ester group is hydrolyzed to yield fexofenadine base.

Other methods of preparing fexofenadine are discussed in U.S. Pat. Nos. 5,578,610, 5,589,487, 5,581,011, 5,663,412, 5,750,703, 5,994,549, 5,618,940, 5,631,375, 5,644,061, 5,650,516, 5,652,370, 5,654,433, 5,663,353, 5,675,009, 5,375,693 and 6,147,216.

The present invention relates to the solid state physical properties of fexofenadine free base prepared by any of these or other methods. These properties can be influenced by controlling the conditions under which fexofenadine free base is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by powder X-ray crystallography, solid state ¹³C NMR spectromety and infrared spectrometry.

U.S. Pat. Nos. 5,738,872, 5,932,247 and 5,855,912, incorporated herein by reference, describe four crystal forms of fexofenadine hydrochloride which were designated Forms I-IV. According to the '872 and related patents, Forms II and IV are hydrates and Forms I and III are anhydrous. Each form was characterized by its melting point, onset of endotherm in the DSC profile, and PXRD. Form I is reported to have a capillary melting point range of 196-201EC, a DSC endotherm with onset between 195-199° C. and a powder X-ray diffraction (“PXRD”) pattern with d-spacings of 14.89, 11.85, 7.30, 6.28, 5.91, 5.55, 5.05, 4.96, 4.85, 4.57, 4.45, 3.94, 3.89, 3.84, 3.78, 3.72, 3.63, 3.07, 3.04, 2.45 Å. Form II is reported to have a capillary melting point range of 100-105° C., a DSC endotherm with onset between 124-126° C. and a PXRD pattern with d-spacings of 7.8, 6.4, 5.2, 4.9, 4.7, 4.4, 4.2, 4.1, 3.7, 3.6, 3.5 Å. Form III is reported to have a capillary melting point range of 166-171° C., a DSC endotherm with onset at 166° C. and a PXRD pattern with d-spacings of 8.95, 4.99, 4.88, 4.75, 4.57, 4.47, 4.46, 3.67, 3.65 Å. In Example 2, Form W is reported to undergo decomposition at 115-116° C. In the general written description, a DSC endotherm with onset at 146° C. is reported. Form IV is reported as having a PXRD pattern with d-spacings of 10.38, 6.97, 6.41, 5.55, 5.32, 5.23, 5.11, 4.98, 4.64, 4.32, 4.28, 4.12, 4.02, 3.83, 3.65, 3.51, 3.46 and 2.83 Å.

The '872 patent discusses methods of interconverting Forms I-IV. Aqueous recrystallization of Form I can be used to produce Form II. Water-minimizing recrystallization or azeotropic distillation of either Form II or Form IV can yield Form I. Form III is reported to be accessible by water minimizing recrystallization of Form II. Crystal digestion of Form III can be used to obtain Form I. Forms II and IV can be obtained directly by sodium borohydride reduction of 4-[4-[4-(hydroxydiphenylmethyl)-1-piperidinyl]-1-oxobutyl]-α,α-dimethylbenzeneacetate as described in Examples 1 and 2.

WO 00/71124 A1, discloses that amorphous fexofenadine hydrochloride can be prepared by lyophilizing or spray drying a solution of fexofenadine hydrochloride. The product is characterized by its IR spectrum and a featureless PXRD pattern.

According to the abstract of WO 01/94313, that publication discloses “a novel crystal form of, alpha-dimethyl-4-[1-hydroxy-4-[4-(hydroxydiphenylmethyl)-1-piperidinyl]butyl]benzeneacetic acid hydrochloride, processes for its preparation and its pharmaceutical use . . . ”

WO 03/039482 (US20030158227), by the same assignee as the present invention, discloses polymorphic forms of fexofenadine base, designated Forms I-VII.

US20030021849, US20020177608 and US20040044038, by the same assignee as the present invention, disclose various polymorphic forms of fexofenadine hydrochloride.

According to the abstract of WO 04/067511, that publication discloses “highly pure fexofenadine and a process for preparing highly pure fexofenadine.”

According to the abstract of WO 05/019175, that publication discloses “anhydrous crystalline fexofenadine hydrochloride Form C, crystalline fexofenadine acetate monohydrate Form D, crystalline fexofenadine acetate dihydrate Form E and crystalline fexofenadine free base monohydrate Form F, processes of preparing the same, pharmaceutical compositions thereof, therapeutic uses thereof and methods of treatment therewith.”

According to the abstract of WO 03/11295, that publication discloses “a novel fexofenadine hydrochloride polymorph.”

Fexofenadine HCl is prepared by reaction of fexofenadine free base with HCl. The purity of the fexofenadine free base used affects the quality of the HCl salt obtained. Thus, there is a need in the art for crystalline forms of fexofenadine free base suitable for conversion to the HCl salt.

### SUMMARY OF THE INVENTION

In one aspect, the present invention provides for a new crystalline form of fexofenadine free base (form VIII), which is characterized by a powder X-ray diffraction pattern with peaks at about 11.9, 17.6, 18.2, 18.6, and 19.4±0.2 degrees two theta.

In another aspect, the present invention provides a process for preparing crystalline fexofenadine free base Form VIII comprising acidifying a basic aqueous solution of fexofenadine in a mixture of water and an organic solvent.

In another aspect, the present invention provides a process for preparing crystalline fexofenadine free base Form VIII comprising the steps of preparing a solution of fexofenadine keto acid in a water miscible organic solvent in the presence of a base and water; adding a reducing agent to the solution to reduce the keto-acid; acidifying reaction mixture obtained from the reduction to precipitate fexofenadine free base and recovering the crystalline form of fexofenadine free base.

### BRIEF DESCRIPTION OF THE FIGURES

FIG. 1 is a PXRD pattern for fexofenadine free base form VIII.

FIG. 2 is a DSC thermogram for fexofenadine free base Form VIII.

FIG. 3 is a TGA thermogram for fexofenadine free base Form VIII.

### DETAILED DESCRIPTION OF THE INVENTION

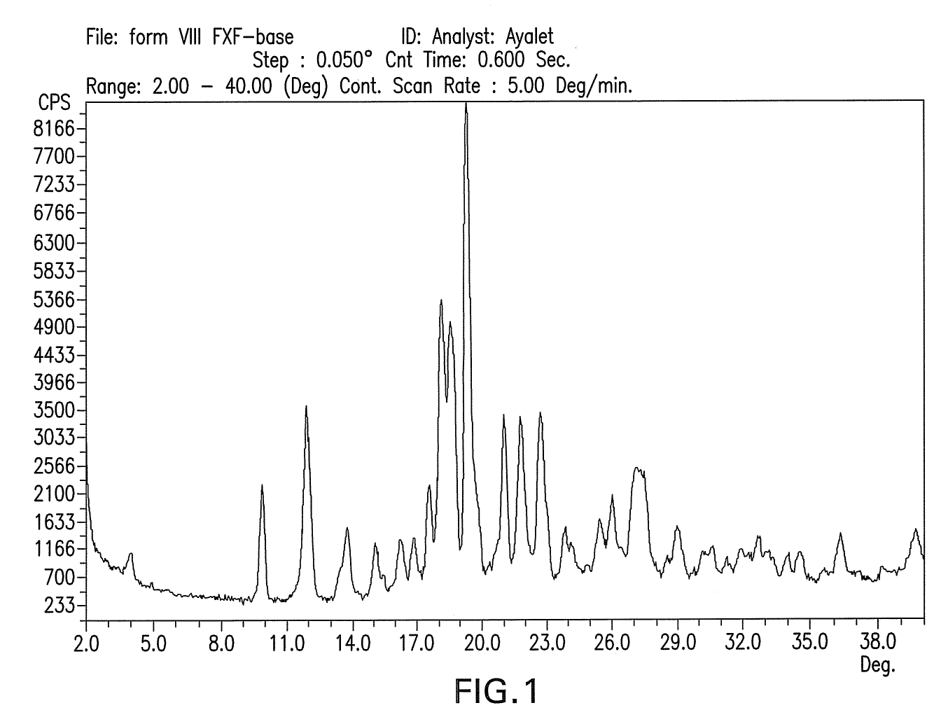

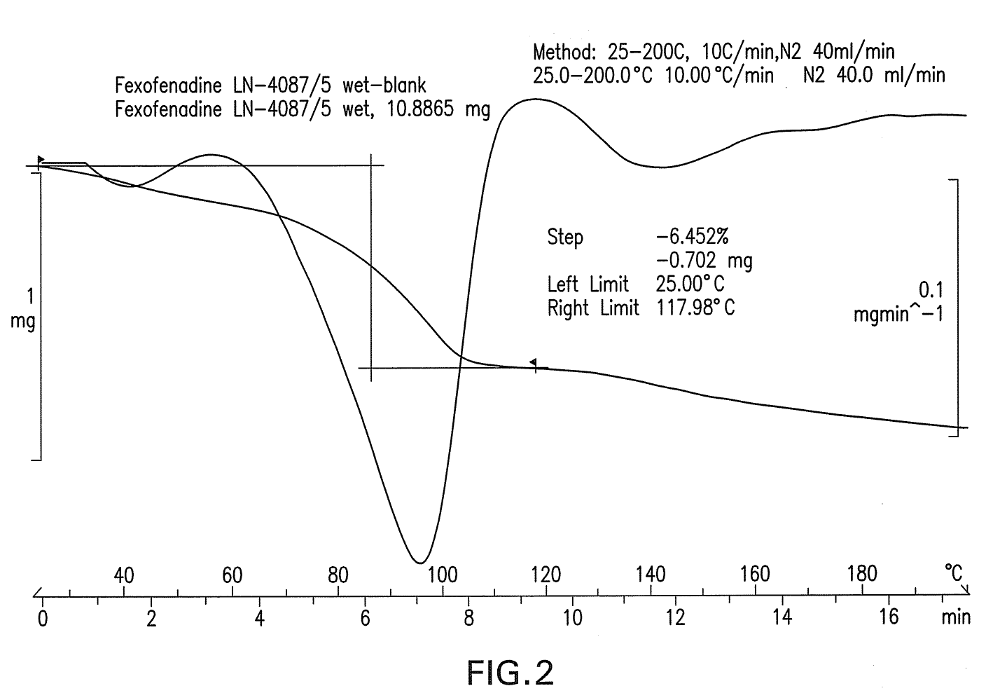

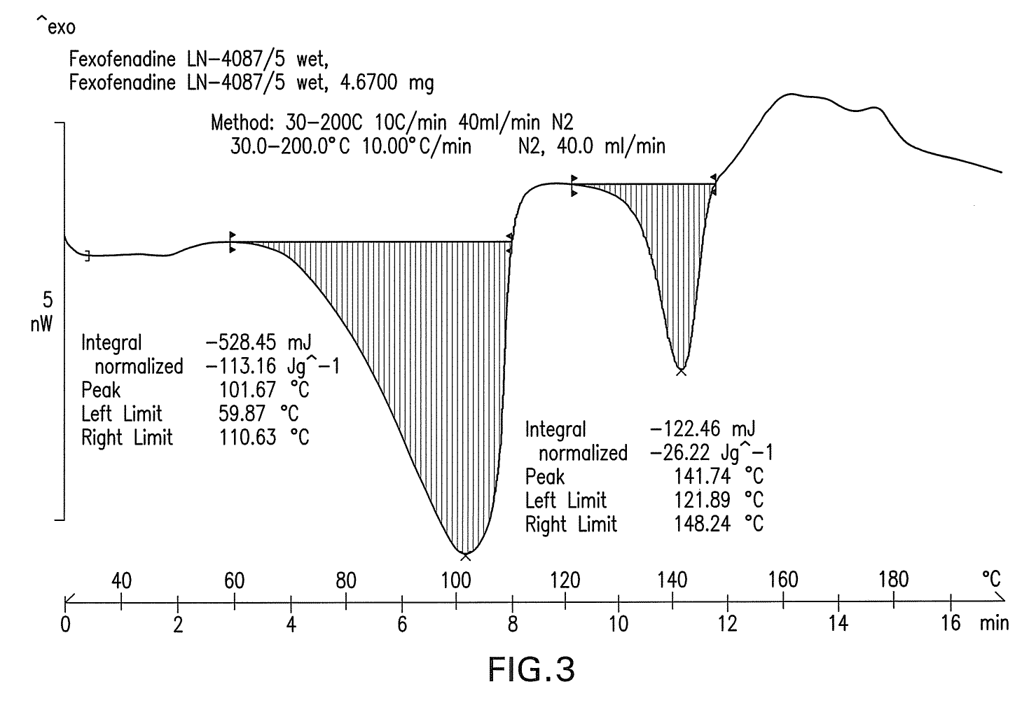

The present invention provides a crystalline form of fexofenadine free base Form (VIII), characterized by an X-Ray diffraction pattern with peaks at 11.9, 17.6, 18.2, 18.6, and 19.4±0.2 degrees two theta. Fexofenadine free base Form VIII may be further characterized by XRD peaks at 9.9, 13.7, 21.0, 21.8, and 22.7±0.2 degrees two theta. The crystalline form may be further characterized by a DSC thermogram with endothermic peaks at about 102° C. and 142° C. The crystalline form may be further characterized by a TGA thermogram showing a weight loss of about 6-7% at a temperature range of about 25-120° C., and about 6-7% water by weight as measured by Karl Fisher. Appropriate PXRD, DSC and TGA figures correspond to FIGS. 1, 2 and 3.

Crystalline fexofenadine Form VIII may be prepared by acidification of a basic aqueous solution of fexofenadine free base containing a mixture of water and an organic solvent. Preferably the organic solvent is a C₁ to C₄ alcohol, more preferably the organic solvent is methanol. Preferably the ratio of the water to alcohol is about 1:1 to about 1:6 by volume.

In the particular example provided, fexofenadine free base form VIII is recovered by preparing a solution of fexofenadine keto acid in a water miscible organic solvent in the presence of a base and water; adding a reducing agent to the solution to reduce the keto-acid and acidifying reaction mixture obtained from the reduction to precipitate fexofenadine free base.

Preferably the water miscible organic solvent is selected from the group consisting of C₁-C₄ alcohols. More preferably, the water miscible organic solvent is methanol.

Preferably the ratio of the water to alcohol is about 1:1 to about 1:6.

Preferably, the base is selected from the group consisting of: NaOH, KOH, NaOMe, NaOtBu and KOtBu. More preferably, the base is NaOH.

When starting with the ketoester, the ketoester dissolves at a high pH of about 13. However, when starting with fexofenadine, a lower pH might be sufficient.

Preferably the reducing agent is selected from the group consisting of: sodium borohydride, potassium borohydride, lithium aluminium hydride (LiAlH₄), and sodium cyanoborohydride (NaBH₃CN). More preferably, the reducing agent is sodium borohydride. When fexofenadine free base is used a starting material, the reducing agent is not necessary. The reducing agent is preferably added at a temperature of about 20° C. to about 35° C.

Preferably, the amount of the reducing agent is higher than about 1 equivalent. More preferably, the amount of the reducing agent is about 1 to about 4 equivalents.

Preferably, an additional amount of water is added during the addition of the acid. Preferably, the total amount of water that is added is about 1.5 liters to about 10 liters per 1 kg of fexofenadine keto acid. More preferably, the total amount of water is about 3 litters per 1 kg of fexofenadine keto acid.

Preferably, the acid is added at a temperature of about less than about 40° C.

Preferably, the acid is preferably selected from the group consisting of HCl, formic acid and acetic acid. More preferably, the acid is acetic acid. The reaction with other acids should be carried out under such conditions that salts do not form.

Preferably, the pH at the end of the reaction is about 5 to about 9, more preferably pH of about 5 to about 6.5. Most preferably, the pH is about 5.

The fexofenadine free base of the present invention may be converted to fexofenadine HCl by reacting the base with HCl. For example, fexofenadine free base Form VIII may be dissolved in water, and contacted with a 36% HCl solution in methanol or THF.

Pharmaceutical compositions of the present invention contain fexofenadine hydrochloride, optionally in mixture with other forms or amorphous fexofenadine and/or active ingredients such as pseudoephedrine. The fexofenadine HCl of these compositions is prepared from the fexofenadine free base of the present invention. In addition to the active ingredient(s), the pharmaceutical compositions of the present invention may contain one or more excipients. Excipients are added to the composition for a variety of purposes.

Diluents increase the bulk of a solid pharmaceutical composition and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form like a tablet may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of non-compacted solid composition and improve the accuracy of dosing. Excipients that may function as glidants include colloidal silicon dioxide, magnesium trisilicate, powdered cellulose, starch, talc and tribasic calcium phosphate.

When a dosage form such as a tablet is made by compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease release of the product form the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate. Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, fexofenadine HCl and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste. Preservatives and chelating agents such as alcohol, sodium benzoate, butylated hydroxy toluene, butylated hydroxyanisole and ethylenediamine tetraacetic acid may be added at levels safe for ingestion to improve storage stability.

A liquid composition according to the present invention may also contain a buffer such as gluconic acid, lactic acid, citric acid or acetic acid, sodium gluconate, sodium lactate, sodium citrate or sodium acetate.

Selection of excipients and the amounts to use may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges as well as liquid syrups, suspensions and elixirs.

An especially preferred dosage form of the present invention is a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filing may be prepared by wet granulation. In wet granulation some or all of the active ingredients and excipients in powder form are blended and then further mixed in the presence of a liquid, typically water, that causes the powders to clump up into granules. The granulate is screened and/or milled, dried and then screened and/or milled to the desired particle size. The granulate may then be tableted or other excipients may be added prior to tableting such as a glidant and or lubricant.

A tableting composition may be prepared conventionally by dry blending. For instance, the blended composition of the actives and excipients may be compacted into a slug or a sheet and then comminuted into compacted granules. The compacted granules may be compressed subsequently into a tablet.

As an alternative to dry granulation, a blended composition may be compressed directly into a compacted dosage form using direct compression techniques. Direct compression produces a more uniform tablet without granules. Excipients that are particularly well suited to direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, only they are not subjected to a final tableting step.

Capsules, tablets and lozenges and other unit dosage forms preferably contain a dosage level of about 60 mg of fexofenadine hydrochloride or base.

### EXAMPLE

### Example 1

Methanol (12.5 liter) was added to a reactor under agitation. Fexofenadine keto acid (based on 5 kg dry) and 2 kg of 47% NaOH and 7.5 liter of water was added to a reactor. Agitation was continued until full dissolution. Sodium borohydride was added gradually to the reactor at 20-35° C. Agitation was continued until end of reaction. Water (7.5 L) was added to the reactor, and acetic acid was added slowly at a temperature of ≦25° C. until pH=5-6. After stirring the suspension for additional 1 hour, the product was filtered. The wet cake was washed with 4 volumes of water and washed three times with two volumes of methanol (each rinse).

### Example 2

Methanol (120 ml), water (6 ml), and 32% HCl solution (10 g) were added to a reactor. The solution was cooled to negative 5° C. under agitation. Fexofenadine base (40 g) was added to the reactor. Agitation was continued until full dissolution was obtained. The solution was cooled under agitation to −12° C. The suspension was stirred for 2 to 16 hours at −12° C. The product was filtered. Pure fexofenadine HCl Form XVI was obtained. The resulting wet cake of fexofenadine HCl Form XVI was dried under vacuum (10 mmHg) at a temperature of 65° C. to 80° C. After 16 hours of drying, pure fexofenadine Form XVI was obtained.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications.

1. A crystalline form of fexofenadine free base characterized by a powder X-ray diffraction pattern with peaks at 11.9, 17.6, 18.2, 18.6, and 19.4±0.2 degrees two theta.

2. The crystalline fexofenadine free base of claim 1, further characterized by XRD peaks at 9.9, 13.7, 21.0, 21.8 and 22.7±0.2 degrees two theta.

3. The crystalline fexofenadine free base of claim 2, wherein the crystalline form has an X-ray powder diffraction diagram as substantially depicted in FIG. 1.

4. The crystalline form of fexofenadine free base of claim 1 having a DSC thermogram with endothermic peaks at about 102° C. and 142° C.

5. The crystalline form of fexofenadine free base of claim 1 having a TGA thermogram showing a weight loss of about 6-7% at a temperature range of about 25-120° C.

6. A process for preparing crystalline fexofenadine free base form of claim 1 comprising acidifying a basic aqueous solution of fexofenadine free base containing a mixture of water and an organic solvent to precipitate the crystalline form and recovering the crystalline form of fexofenadine free base.

7. The process of claim 6, wherein the organic solvent is a C₁ to C₄ alcohol.

8. The process of claim 7, wherein the alcohol is methanol.

9. The process of claim 6, wherein the acid is acetic acid.

10. (canceled)

11. (canceled)

12. A process for preparing crystalline fexofenadine free base of claim 1 comprising the steps of preparing a solution of fexofenadine keto acid in a water miscible organic solvent in the presence of a base and water; adding a reducing agent to the solution to reduce the keto-acid; acidifying reaction mixture obtained from the reduction to precipitate fexofenadine free base and recovering the crystalline form of fexofenadine free base.

13. The process of claim 12, wherein the water miscible organic solvent is selected from the group consisting of C₁-C₄ alcohols.

14. The process of claim 13, wherein the C₁-C₄ alcohol is methanol.

15. The process of claim 12, wherein the ratio of the water to the water miscible organic solvent is about 1:1 to about 1:6.

16. The process of claim 12, wherein the base is selected from the group consisting of: NaOH, KOH, NaOMe, NaOtBu and KOtBu.

17. The process of claim 16, wherein the base is NaOH.

18. The process of claim 12, wherein the reducing agent is selected from the group consisting of: sodium borohydride, potassium borohydride, lithium aluminium hydride (LiAlH₄), and sodium cyanoborohydride (NaBH₃CN).

19. The process of claim 18, wherein the reducing agent is sodium borohydride.

20. The process of claim 12, wherein the reducing agent is added to the solution at a temperature of about 20° C. to about 35° C.

21. The process of claim 12, wherein the amount of the reducing agent is higher than about 1 equivalent.

22. The process of claim 21, wherein the amount of the reducing agent is about 1 to about 4 equivalents.

23. The process of claim 12, wherein the acid is selected from the group consisting of HCl, formic acid and acetic acid.

24. The process of claim 23, wherein the acid is acetic acid.

25. The process of claim 12, wherein acidifying is carried out to a pH of about 5 to about 9.

26. The process of claim 25, wherein acidifying is carried out to a pH of about 5 to about 6.5.

27. The process of claim 26, wherein acidifying is carried out to a pH of about 5.

28. The process of claim 12 further comprising adding water during the addition of the acid.

29. (canceled)

30. (canceled)
